(19) United States
(12) Patent Application Publication
(10) Pub. No.: US 2009/0232749 A1
(43) Pub. Date: Sep. 17, 2009

(54) Compositions for the acute and/or long term treatment of periodontal diseases

(75) Inventor: Richard Jay Adler, Mallorca (ES)

Correspondence Address: HOFFMANN & BARON, LLP, 6900 JERICHO TURNPIKE, SYOSSET, NY, 11791, US

(73) Assignee: EUROPEAN NATURAL PRODUCTS, Amstelveen (NL)

(21) Appl. No.: 11/720,768

(22) PCT Filed: Nov. 24, 2005
(86) PCT No.: PCT/IB05/03534

§ 371 Date: Dec. 20, 2007

(30) Foreign Application Priority Data
Dec. 3, 2004 (IB) PCT/IB2004/004398

(51) Int. Cl. A61K 8/97; A61Q 11/00; A61P 31/04; A61P 31/10

(52) U.S. Cl. 424/58

(57) ABSTRACT

Composition comprising a mixture of radix polygoni multiflori, Fructus Corni, cuscuta japonica, rehmannia glutinosa, licorice, asparagine, Derla andrographis and a composition comprising a mixture of Salvida persica, achyranthes aspera, spilanthes acmela, clove, picus Bengalensis, acacia nilotioca resen, eucalyptus, mint, green tea, bamboo silica for the acute and/or long term treatment of of microbial infections, in particular of oral pathogenic micro-organisms, in particular dental caries, periodontosis, gingivitis, gum disease, gum bleeding and/or plaque reduction.

### FIELD OF THE INVENTION

This invention relates generally to the field of herbs, specifically to herbs useful for the treatment or prevention of microbial infections, in particular periodontal diseases. The invention also relates to the use of compositions for the acute and/or long term treatment or prevention of periodontal diseases and to a method for preparing the compositions.

### BACKGROUND OF THE INVENTION

Modern medical science is constantly searching for new and more powerful agents to prevent, treat or retard bacterial and viral infections and cure the diseases they cause. Bacterial and viral infections of humans and domestic animals cost billions of dollars annually. Vast sums of money are spent each year by pharmaceutical companies to identify, characterise, and produce new antibiotics and antivirals to combat the emerging drug resistant strains which have become a serious problem. Reliable prophylactic treatments for disease prevention are also of major interest.

Specifically, periodontal disease and dental caries are of major public health and economic interest world-wide. It is now widely recognised that both of these oral diseases are caused by bacteria which grow in masses on the teeth and in the gingival and subgingival areas. A commonly used descriptive term for these bacterial masses is “dental plaque”. In the case of periodontal disease, it has been reported that dental plaque bacteria, growing in the area where the teeth and gingival tissues meet, cause an inflammation of the gingiva called “gingivitis”. This is characterised by swollen, edematous gingiva (“gums”) which are reddened and bleed easily. If plaque removal is inadequate, gingivitis may progress to “periodontitis” or periodontal disease in some individuals. Periodontitis generally is characterised by a chronic inflammation of the tissues around the teeth, which leads to a resorption of supporting bone. Periodontal disease is the leading cause of tooth loss among adults.

Dental caries (cavities) are also caused by bacteria, with mutans Streptococcus being the principal etiologic agent. Dental caries is a prevalent and costly disease throughout the world. The latest report by NIH indicated that 49% of 12-year-old and 79% of 17-year-old children in the USA have dental caries. A very high percentage of the elderly also have tooth decay manifest as root caries.

Tooth decay is mainly caused by a group of cariogenic Gram-positive bacteria such as Streptococcus mutans. Given a suitable carbohydrate nutrient (simple dimer sugars like sucrose), these bacteria produce insoluble glucans and acids in dental plaque. The glucans produced by S. mutans are very sticky, enabling it to adhere to the tooth's surface while the acids attack the tooth's mineral structure causing demineralisation that may lead to cavitation.

The prevention of dental plaque or the removal thereof has long been the focus of development, with the ultimate goal of inhibiting both caries and periodontal diseases. While the formation of dental plaque can be inhibited to a certain extent by brushing the teeth at frequent intervals, brushing alone is not sufficient to effectively prevent the formation of dental plaque or remove substantially all of the dental plaque that has formed on the teeth. Since brushing alone is often not sufficient to prevent dental caries or periodontal disease due to the nature of the pathogenic plaque bacteria, chemical methods using anti-bacterials such as chlorhexidine, benzalkonium chloride, and cetylpyridinium chloride have been proposed.

The use of so-called Chinese herbal medicine in the treatment of these diseases has been the subject of prior research. US2003/091517, WO03/099110, EP1415646 and WO03/094941 all describe prevention or treatment against the microbial infections underlying the periodontal diseases.

Periodontitis is a very widespread disease, often starting in the 20 years old period and has a relentless, chronic course. Current treatments are poor, with great discomfort to patients and generally with poor results. Accordingly, there is a need in the art to provide further compositions or products useful for treating or preventing microbial conditions, e.g., oral microbial conditions such as periodontal disease and dental caries.

### SUMMARY OF THE INVENTION

The present invention is based on the discovery that a pool of natural herbs or the combinations thereof have anti-microbial activity, e.g., anti-bacterial, anti-fungus activity. It has further been found that certain specific mixtures of these herbs or extracts thereof expressing this activity are suitable for the acute treatment or prevention and/or for the long-term treatment or prevention. It has also been found that when the compositions are prepared from extracts that have been obtained using specific extraction methods, an extra effect is obtained in terms of improved or prolonged activity. Accordingly, the present invention provides compositions of herbal combinations useful for treating or preventing microbial conditions, e.g., oral microbial conditions such as periodontal disease and dental caries. The present invention also provides methods of using herbs and the combinations thereof to treat or prevent microbial conditions, e.g., oral microbial conditions such as periodontal disease and dental caries. The present invention also provides for the use of the compositions for the preparation of a medicament for the treatment or prevention of microbial conditions, e.g., oral microbial conditions such as periodontal disease and dental caries.

In one embodiment, the present invention provides a first composition comprising a mixture of at least two components selected from the group consisting of radix polygoni multiflori, Fructus Corni, cuscuta japonica, rehmannia glutinosa, licorice, asparagine and Derla andrographis.

In another embodiment, the present invention provides a composition comprising a mixture of at least three components selected from the group consisting of radix polygoni multiflori, Fructus Corni, cuscuta japonica, rehmannia glutinosa, licorice, asparagine and Derla andrographis.

In yet another embodiment, the present invention provides a composition comprising a mixture of at least four, five or six components selected from the group consisting of radix polygoni multiflori, Fructus Corni, cuscuta japonica, rehmannia glutinosa, licorice, asparagine and Derla andrographis.

In yet another preferred embodiment, the present invention provides a composition comprising a mixture of radix polygoni multiflori, Fructus Corni, cuscuta japonica, rehmannia glutinosa, licorice, asparagine and Derla andrographis.

In one embodiment, the present invention provides a second composition comprising a mixture of at least at least one, two, three four, five, six, seven, eight or nine components selected from the group consisting of Salvida persica, achyrahthes aspera, spilanthes acmela, clove, picus Bengalensis, acacia nilotioca resen, eucalyptus, mint, green tea and bamboo silica.

In still another embodiment, the present invention provides a composition comprising a mixture of at least one, two, three four, five or six components selected from the group consisting of radix polygoni multiflori, Fructus Corni, cuscuta japonica, rehmannia glutinosa, licorice, asparagine, Derla andrographis with at least one, two, three four, five, six, seven, eight or nine components selected from the group consisting of Salvida persica, achyranthes aspera, spilanthes acmela, clove, picus Bengalensis, acacia nilotioca resen, eucalyptus, mint, green tea, bamboo silica

In yet another embodiment, the present invention provides a method of treating a microbial infection comprising administering to a subject in need of such treatment the first composition and/or the second composition.

In another embodiment, the present invention provides a method of preventing a microbial infection. The method comprises contacting a composition with an area susceptible to a micro-organism causing the microbial infection, wherein the composition is the first composition and/or the second composition.

### DESCRIPTION OF THE PREFERRED EMBODIMENTS

The first composition has been found particularly active in the acute treatment of periodontitis. Acute treatment as used herein is a relative term and means the phase of treatment wherein the disease is substantially reduced or mitigated to the extent that the patient is considered cured. In certain embodiments, the micro-organisms are reduced in amount by 50%, 60%, 70%, 80% compared to the start of the treatment. In certain embodiments, the micro-organisms are reduced in amount by 90%, preferably 95%, more preferably 96%, most preferably 97%. In especially preferred embodiments, the micro-organisms are reduced in amount by 99 or 99.9% compared to the start of the treatment. In certain embodiments, the effectiveness of the treatment is measured based on the parameters Gingival index, bleeding index, Periodontal probing depths, attachment levels and/or plaque index. Effectiveness in measured clinically using a placebo group. Effectiveness is expressed in % reduction of the relevant index compared to the placebo group. In certain embodiments, the relevant index is reduced by 10%, 20% or 30%, preferably by 40, 50 or 60%, more preferably by 70 80 or 90%. When seen in time, the acute phase is usual one or two weeks, sometimes three or four weeks and in certain occasions five or six weeks. Preferably one month. Typically a treating dentist or physician can readily assess, based on the relative parameters whether the acute phase is over.

The second composition has been found particularly active in the long-term treatment of periodontitis. Long-term treatment as used herein is a relative term and means the phase of the treatment following the acute phase. This treatment may take from several months (more than 2 months or more than 3 months) up to a life long treatment. Conventional treatments only take care of the acute phase and do not realise that continuing treatment is necessary to adequately cure the disease. If not properly treated after the acute phase, the disease will return. Very often continuous treatment or continuous prevention is needed. The second composition of the present invention provides such a long-term treatment or prevention.

Advantageously, the disease is first treated with the first composition and subsequently with the second composition. In certain embodiments, the first and second composition may be combined to provide a single treatment or can be in the form, of a kit of parts, one part for the acute phase and one part for the long-term phase.

In certain embodiments, the first composition comprises Fleece-flower (radix polygoni multiflori), Dried rehmannia (rehmannia glutinosa), Licorice, Dogwood fruit (Fructus Corni), Dodder seed (cuscuta japonica), Derla Andrographis (Derla andrographis), Asparagine (amide transferase). In certain embodiments, the components can be present in independent amounts of about 50 to about 250 mg on a total weight per dose of about 800 mg to about 1200 mg. Thus, the components can be present in an independent amount from 6 to 30 parts by weight.

In certain embodiments, the rehmannia radix (the stem dried root of rehamnnia glutinose of the scrophularie family) is used. In certain embodiments, Andrographidis herbal (the entire plant of the andropgraphis piniculata of the acanthaceae family) is used.

In certain embodiments, the second composition comprises Salvadora Persica in the form of the leaf and roots extract, Achyranthes Aspera in the form of a roots extract, Spilanthes Acmela root powder, preferably not the extract, Clove, extract, Picus Bengalensis in the form of aerial roots extract, Acacia Nilotica Resen extract, Eucalyptus in the form of a leaf extract, Mint extract, Green Tea Extract containing 40% Catechins, Bamboo Silica.

In certain embodiments the second composition comprises:

| | amount (parts by |
|---|---|
| Name | weigth) |
| Salvadora Persica | 200 |
| Achyranthes Aspera | 50 |
| Spilanthes Acmela | 25 |
| Clove | 25 |
| Picus Bengalensis | 50 |
| Acacia Nilotica Resen | 50 |
| Eucalyptus | 25 |
| Mint | 50 |
| Green Tea Extract | 75 |
| Bamboo Silica | 50 |

The present invention relates in general to herbs and combinations thereof useful for treating or preventing microbial conditions. It is the discovery of the present invention that certain herbs and combinations thereof have anti-microbial activity, e.g., anti-bacterial, anti-fungal activity, or ability of interrupting bacterial quorum sensing. Accordingly, the present invention provides compositions and methods of using the compositions for treating or preventing microbial conditions, e.g., oral microbial conditions such as periodontal disease and dental caries.

The herbs in the composition of the present invention can have any weight ratios suitable for providing the composition with an anti-microbial activity. One skilled in the art can readily determine such suitable weight ratios by testing anti-microbial activity of compositions of different weight ratios in routine bioassays. Generally the weight ratio for each herb of the composition may vary from about 1 to about 10, e.g., (1-10):(1-10), (1-10):(1-10):(1-10), and (1-10):(1-10):(1-10):(1-10) and so on for each herb in the composition. Preferably, the weigh ratio may vary from about 1 to about 5, e.g. (1-5):(1-5), (1-5):(1-5):(1-5), (1-5):(1-5):(1-5):(1-5), (1-5):(1-5):(1-5):(1-5):(1-5), (1-5):(1-5):(1-5):(1-5):(1-5):(1-5), (1-5):(1-5):(1-5):(1-5):(1-5):(1-5):(1-5). More preferably the weight ratio may vary from about 1 to about 2, e.g. (1-2):(1-2), (1-2):(1-2):(1-2), (1-2):(1-2):(1-2):(1-2), (1-2):(1-2):(1-2):(1-2):(1-2), (1-2):(1-2):(1-2):(1-2):(1-2):(1-2), (1-2):(1-2):(1-2):(1-2):(1-2):(1-2):(1-2).

In one embodiment, about the same amount of each herb is used in the composition of the present invention, e.g., about equal ratio for each herb such as 1:1, 1:1:1, or 1:1:1:1 etc.

In certain preferred embodiments, the first composition may comprise the following weight ratio's of each herb, by approximation, i.e. a variation of about at least 30%, preferably about at least 20%, more preferably about at least 10% and even more preferred about at least 5% variation is allowed, without departing from the gist of the invention.

| | | | | |
|---|---|---|---|---|
| Comp. I | II | III | IV | V |

| | | | | | |
|---|---|---|---|---|---|
| Fleece-flower | 1-15 | 3-12 | 6-10 | 7-9 | 8 |
| Dried rehmania | 1-15 | 3-12 | 6-10 | 7-9 | 8 |
| Licorice | 0.5-15 | 0.6-5 | 0.8-2 | 0.9-1.1 | 0.94 |
| Dogwood fruit | 1-15 | 1-12 | 2-10 | 3-6 | 4 |
| Dodder seed | 1-15 | 1-12 | 1-10 | 3-4 | 2 |
| Derla Andrographis | 1-15 | 1-12 | 1-10 | 3-4 | 2 |
| Asparagine | 1-15 | 1-12 | 2-10 | 4-5 | 4 |

In certain preferred embodiments, the second composition may comprise the following weight ratio's of each herb, by approximation, i.e. a variation of about at least 30%, preferably about at least 20%, more preferably about at least 10% and even more preferred about at least 5% variation is allowed, without departing from the gist of the invention.

| | VI | VII | VIII | IX | X |
|---|---|---|---|---|---|
| Salvadora Persica | 1-15 | 5-12 | 6-11 | 7-10 | 8 |
| Achyranthes Aspera | 0.5-15 | 0.8-10 | 0.9-5 | 1-4 | 2 |
| Spilanthes Acmela | 0.5-15 | 0.7-10 | 0.8-5 | 0.9-1.1 | 1 |
| Clove | 0.5-15 | 0.7-10 | 0.8-5 | 0.9-1.1 | 1 |
| Picus Bengalensis | 0.5-15 | 0.7-10 | 0.8-5 | 1-4 | 2 |
| Acacia Nilotica Rese | 0.5-15 | 0.7-10 | 0.8-5 | 1-4 | 2 |
| Eucalyptus | 0.5-15 | 0.7-10 | 0.8-5 | 0.9-1.1 | 1 |
| Mint | 0.5-15 | 0.7-10 | 0.8-5 | 1-4 | 2 |
| Green Tea Extract | 0.5-15 | 0.8-10 | 1-8 | 2-5 | 3 |
| Bamboo Silica | 0.5-15 | 0.7-10 | 0.8-5 | 1-4 | 2 |

A typical formulation according to the invention contains a composition of the herbs as outlined herein in an amount of at least 10 milligrams, preferably at least 50 mg, more preferable at least 100 mg, even more preferable at least 250 mg. In certain preferred embodiments, a typical formulation contains from about 500 mg per dose, preferably at least 750 mg per dose, more preferable at least 1000 mg per dose, and especially preferred more than 1500 mg per dose.

On the other end, the formulation typically contains not more than 2000 mg, 1500 mg, 1250 mg, 1000 mg, 750 mg, 500 mg, 250 mg, 100 mg, 50 mg.

These ranges of amounts in the formulation apply to the first compostion, the second composition or the combination of the first and second composition.

According to another feature of the present invention, the compositions according to the invention can be combined with other known anti-G<+> bacterial agent,anti-G<−> bacterial agent, anti-fungus agent and can be used in combination with the comprehensive anti-microbial compositions of the present invention. The agents used for the comprehensive anti-microbial composition of the present invention can be any entity having the desired activity. For example, the agents used for the comprehensive anti-microbial composition of the present invention can be chemical compounds, polypeptides, polynucleotides, small molecules, recombinant materials, herbs, natural substance, or any synthetic substances.

The composition of the present invention can also include one or more other non-active ingredients, e.g., ingredients that do not interfere with the function of the active ingredients. For example, the composition of the present invention can include a suitable carrier or be combined with other therapeutic agents.

A suitable carrier can be an aqueous carrier including any safe and effective materials for use in the compositions of the present invention. In one embodiment, an aqueous carrier can be used for the compositions of the present invention in oral formations. In certain embodiment, the compositions of the present invention can be formulated with, without limitation, thickening materials, humectants, water, buffering agents, abrasive polishing materials, surfactants, titanium dioxide, flavor system, sweetening agents, coloring agents, antioxidants, adjuvants, preservatives, stabilisers, homogenising, agents, texturising agents, soothing agents and mixtures thereof.

Pharmaceutically acceptable salts can also be used in the composition, for example, mineral salts such as sodium or stannous fluorides, or sulfates, as well as the salts of organic acids such as acetates, proprionates, carbonates, malgnates, or benzoates. The composition can also contain liquids, e.g., water, saline, glycerol, and ethanol, as well as substances, e.g., wetting agents, emulsifying agents, or pH buffering agents.

According to another feature of the present invention, the compositions of the present invention can be used to treat or prevent microbial growth or infection, e.g., inhibit the activity of bacteria or fungi in vivo or in vitro. For example, the compositions of the present invention can be used to inhibit microbial flora, especially microbial flora associated with dental structures, e.g., tooth surface or subsurface or caries, e.g., microbial flora associated with demineralized areas, white spots, pits,and fissures. In one embodiment, the compositions of the present invention can be used to inhibit microorganisms including, without limitation, S. mutans, S. sobrinus, L. acidophilus, L. casei, L. plantarum, A. naeslundii, A. viscosus, Actinobacillus actinomycetemcomitants, Porphyromonas gingivalis, Fusobacterium nucleatum, Treponema denticola, Bacteroides forsythus, Candidas albicans, C. glabrata, C. guilliemondii, C. kefyr, C. krusei, C. stellatoidea and C. tropicalis.

In another embodiment, the composition of the present invention can be used to inhibit the activity of cariogenic bacteria, including without limitation, Mutans streptococci, lactobacilli and actinomyces, e.g., S. mutans, S. sobrinus, A. viscosus, A. naeslundii, L. acidophilus, L. casei, and L. plantarum. In yet another embodiment, the composition of the present invention can be used to inhibit the activity, of fungi, e.g., Candidas albicans, C. glabrata, C. guilliemondii, C. kefyr, C. krusei, C. stellatoidea and C. tropicalis.

According to another feature of the present invention, it provides a method of inhibiting the activity of micro-organisms from one or more species or preventing a microbial infection by contacting one or more compositions of the present invention with the micro-organisms. The present invention also provides a method for treating or preventing a microbial infection by administering to a subject in need of such treatment an effective amount of one or more compositions, of the present invention. The subject in need of such treatment can be any suitable subject, e.g., a human or an animal including a domestic animal such as a horse, dog, or cat. The microbial infection can be any infection caused by one or more, micro-organisms of one or more species including without limitation microbial infections associated with multi-species biofilms.

In generally, an effective amount of the compositions to be administered can be determined on a case-by-case basis. Factors to be considered usually include age, body weight, stage of the condition, other disease conditions, duration of the treatment, and the response to the initial treatment.

According to another embodiment of the present invention, the compositions of the present invention are used to treat or prevent cariogenic organism infections, e.g., S. mutans infection associated with dental caries, including, without limitation, tooth surface or subsurface associated with demineralised areas, white spots, pits, and fissures. One or more compositions of the present invention can be prepared as additives to food, oral hygiene product, or any products having direct contact to an oral environment, especially an oral environment susceptible to dental caries or periodontal diseases. For instance, to treat, or prevent dental caries or periodontal diseases compositions of the present invention can be formulated into a baby formula, mouthwash, lozenges, gel, varnish, toothpaste, toothpicks, tooth brushes, or other tooth cleansing devices, localised delivery devices such as sustained release polymers or microcapsules, oral irrigation solutions of any kind whether mechanically delivered or as oral rinses, pacifiers, and any food including, without limitation, chewing gums, candies, drinks, breads, cookies, and milk.

The invention further relates to a method for the preparation of an extract from the components of the first and or second composition comprising supercritical carbon dioxide liquid extraction. It was observed that the extract from the components obtained in this manner expresses an increased activity compared to conventionally obtained extracts (i.e. from alcohol or water).

### EXAMPLES

The following examples are intended to illustrate but not to limit the invention in any manner, shape, or form, either explicitly or implicitly. While they are typical of those that might be used, other procedures, methodologies, or techniques known, to those skilled in the art may alternatively be used.

The purpose of this study was to compare the effect of certain nutritional and plant-derived herbal supplement (PerioTab) and a placebo tablet in the reduction of gingivitis, bleeding, probing depths, plaque accumulation and attachment levels, on a two month, two-cell, randomized, parallel, clinical trial for patients with Type II periodontal disease.

Each cell consisted of 30 and 32 subjects for a total of 62 participants. Demographic information and medical history were randomly collected from each subject. Oral examinations of soft tissues and indices of gingivitis, bleeding, pocket depths and attachment levels were performed at baseline, one and two-months. GI, BI, AL, PI and PD were analyzed using a one-way ANOVA, one-way repeated after factor ANOVA and paired t-test procedures.

Overall the data shows a reduction in the gingivitis, bleeding on probing, plaque accumulation for the PerioTab but no significant changes in the attachment levels and pocket depth reduction. For the placebo, there was little reduction in the gingivitis, bleeding index, pocket depths, attachment level and plaque, accumulation at any of the time periods.

PerioTab was the most effective at reducing gingivitis, bleeding and plaque accumulation, which were significantly different from the Placebo (p<0.0001). Neither of the two products was effective at reducing the Attachment Levels or pocket depths for either males or females.

It is concluded that PerioTab is effective at reducing gingivitis, bleeding and plaque accumulation over a short period of time and should be used as an adjunct to periodontal therapy.

Purpose

To evaluate the benefit of certain nutritional and plant-derived herbal ingredients in the treatment of periodontal disease.

Introduction

Among all the recommendations for the maintenance of gingival and periodontal health, few have focused on the value of nutritional supplements. The purpose of this study is to compare the effect of certain nutritional and plant derived natural herbal ingredients (PerioTab) and a placebo tablet in the reduction of gingivitis, bleeding, probing depths and attachment levels, on a two month, two-cell, randomized, parallel, clinical trial for patients with Type II periodontal disease.

Fleece-Flower Root (Radix Polygoni Multiflori)

This herb is the root tuber of Poly-gonum multiflorum Thunb (family Poly-gonaceae), which is produced in all parts of China. Bitter, sweet and astringent in flavor, slightly warm in property, acting on the liver and kidney channels. It claims to replenish the vital essence and blood, curing malaria, clearing away toxins, moistening the intestines and relieving constipation.

Dogwood Fruit (Fructus Corni)

This herb is the pulp of the ripe fruit of Cornus officinalis Sieb. etZucc. (family Cor-naceae), which is produced mainly in Zhejiang, Anhui, Henan and Shanxi provinces. After collection in the late autumn, the fruit is baked over slow fire or scalded slightly in boiling water, and then dried in the sun or baked dry for use after removal of the fruit stone.

It claims to nourish the liver and kidney and induces astringency to arrest incessant and excessive loss of the body fluid. It serves as an antioxidant.

Dodder Seed

The leafless Dodder plant is native to China and Japan. Also known as Cuscuta japonica, it is from the family Convolvulaceae. Dodder seed is one of the chief ingredients in the famous Ming Dynasty formula known as ‘Seven Treasures’. Consisting of Dodder seed, Fo Ti, Lycium, Dong Quai, Poria, Achyranthes and Psoralea, this formula has been used for centuries as a superior antiaging tonic to help rejuvenate the body and prolong life. Currently, it is available with the addition of Ligustrum fruit and Astragalus seed for increased energy to support today's stressful, lifestyle.

This improved Seven Treasures formula, with the addition of Ligustrum fruit and Astragalus seed, claims to help nourish the back, knees, hair, skin, eyes, ears, teeth and bones while enhancing sexual vitality and promoting longevity.

Asparagine

Asparagine is an α-amino acid found in proteins. Asparagine is classified as an amide because it is an amide derivative of aspartic acid. It is one of the 20 amino acids commonly found in animal proteins. Only the 1-stereoisomer participates in the biosynthesis of mammalian proteins. Its structure is identical to that of the amino acid aspartic acid, except that the latter compound's acidic side-chain carboxyl group has been coupled with ammonia, yielding an amide.

Like glutamine, asparagine is important in the metabolism of toxic ammonia in the body. The relatively unreactive, neutral amide group in the side chain of asparagine confers no special properties upon this amino acid once it is included within a protein by two peptide bonds. Asparagine is not essential to the human diet, since it can be synthesized from aspartic acid. The first amino acid to be isolated from its natural source, asparagine was purified from asparagus juice in 1806; proof of the occurrence of this amino acid in proteins was finally obtained in 1932.

Licorice

In Sanskrit, it is called sweet stalk. The Greeks named it sweet root. And the Chinese, who may have known about it the longest, dubbed it gancao, which means sweet grass.

The industry uses it for flavoring cigarettes, cigars, and chewing tobacco. Beverage makers use licorice as a foaming agent. Pharmaceutical companies use the sweetness of licorice to mask the taste of bitter drugs.

Traditional Chinese medicine extensively calls for licorice as a herbal healing agent. Europeans use it as a soothing agent in cough suppressants and to help heal ulcers. And in early Western medicine, licorice was found to relieve the symptoms of Addison's disease.

Though licorice lovers in the U.S. rarely eat enough glycyrrhizic acid to get a blood-pressure boost, European physicians are now debating whether moderate doses of natural licorice have a significant hypertensive effect. Glycyrrhizic acid, which makes up from 4 to over 20% of the root, is not the only biologically active molecule. About 300 different, polyphenols, which make up 1 to 5% of the root, are suspected antioxidants, perhaps even cancer-fighting compounds.

Rehmanniae Radix

It is the stem-dried root of Rehmannia Glutinosa of the Scrophularie family. Its principal constituent is Stachyose. Other ingredients include D-glucose, D-fructose, sucrose, raffinose, manninotriose, verbascose, mannitol and vitamin A.

It claims to nourish blood and sperm, supplements kidneys and liver. The extract is cardiotonic to an exhausted heart, acting directly on heart muscles. Because it is cardiotonic and dilates renal blood vessels it produces diuretic action.

Andrographidis Herbal (Dried Andrographis)

It is the entire plant of of Andrograhis piniculata or the Acanthaceae family. The principal constituents are Andrographolide, meoandrographolide and paiculide.

It claims to dispel heat and removes toxins. Has an antibacterial effect (It inhibits Diplococcus pneumoniae and other bacteria) and has an antiviral effect by delaying the embryonic renal cells caused by virus ECHO. It is used for infectious diseases, tonsillitis, bronchitis, pneumonia, acute enteritis, red dysentery, urethritis, nephritis, pustular dermatitis, and purulent otitis media.

Study Design

A sixty-day, two-cell, randomized, parallel, clinical trial for patients with Type II periodontal disease was conducted. Each cell consisted of 30-33 subjects for a total of 63 participants. Qualified subjects in one cell received a vitamin tablet (PerioTherapy) with the active ingredients, while the other cell received a placebo tablet. Each subject took take the tablet twice a day for 7 days.

The treatment tablet (PerioTherapy) and the placebo tablet were provided by the sponsor with identical packaging which was labeled as A=Placebo or B=PerioTab, so that neither the subject nor the investigators knew the treatment. Clinical parameters that were evaluated included: 1) gingival index, 2) bleeding index, 3) periodontal probe depth and 4) attachment levels. All tests were carried out using modified indices. Clinical measures of efficacy were made at baseline, thirty and sixty days.

### Composition of PerioTab (500 mg):

| Name: | Relative Amount |
|---|---|
| Fleece-flower | 896 |
| Dried rehmanria | 896 |
| Licorice | 105 |
| Dogwood fruit | 448 |
| Dodder seed | 224 |
| Derla Andrographis | 224 |
| Asparagine (amide transferase) | 448 |

### Composition of Placebo Tablet:

The placebo tablet was composed of a gelatin capsule and powdered lactose.

Subjects were asked to take the assigned tablet following instructions provided by the sponsor. To monitor compliance, subjects maintained daily logs of when they took the tablet.

As mandated by California Law and the Institutional Review board, the study was conducted in accordance with ICH guidelines for Good Clinical Practices (GCP).

Subjects Enrollment and Recruitment

The Center for Dental Research had approximately 82 potential subjects. Sixty subjects were selected for the study. An additional three subjects were enrolled to compensate for early withdrawals or terminations.

| | |
|---|---|
| Total # of subjects screened: | 82 |
| Total # of subjects that passed screening: | 65 |
| Total # of subjects on product: | 63 |
| Total # of subjects dropped: | 1 |
| Total # of subjects that completed the study: | 62 |

Table 1 indicates the breakdown of the participant subjects by race, gender and age.

Inclusion/Exclusion Criteria

The screening process began on Oct. 14, 2003 and the study was completed on Dec. 18, 2003. All subjects were enrolled and on product by Oct. 15, 2003. The last subject was evaluated Dec. 18, 2003.

### Inclusion Criteria:

Subjects were eligible to enter the study if they meet the following criteria:
1. Able to read, understand and sign the informed consent.
2. Be in good general health as evidenced by their medical history.
3. Be between the ages of 25 and 75.
4. Have a minimum of 20 natural teeth, including at least 5 maxillary and 5 mandibular anterior teeth, but excluding third molars.
5. Capable of understanding and following oral instructions as to the plan and scope of the study.
6. Have a mean gingival index score of 2
7. Had mild to moderate periodontitis in at least one quadrant of the mouth, each of which contain at least three pockets which measured 4 to 5 mm, and bled to gentle probing. None of the qualifying sites should exceed a 7-mm periodontal pocket depth. Periodontal pockets in which the depth of the pocket corresponds to the apex to the tooth, as in a possible endodontic/periodontic condition, were not treated or evaluated.

### Exclusion Criteria:

Subjects with one or more of the following conditions were excluded from the study:
1. Be on chronic concomitant medications that would affect his/her soft tissue health (e.g. Dilantin, steroids, etc.).
2. The need for prophylactic antibiotic coverage for dental procedures.
3. Individuals with a chronic infectious disease with oral manifestations that would jeopardize his/her health or that of other health care providers.
4. Suspected or known allergy to acrylic resins or latex gloves.
5. Pregnant or nursing women.
6. Has a cardiac pacemaker.
7. Subjects not expected to demonstrate compliance, such as extensive travel commitments, lack of transportation, etc.
8. Smokers

Clinical Procedures

### 1.-Data Collection and Subject Measurements

Clinical record forms (C.R.F.) were prepared for data collection for each subject. The investigators according to the following schedule completed the forms:
a.-Baseline Examinations:
1.-Informed consent was signed
2.-Medical and health survey was taken
3.-Oral Soft Tissue examination
4.-Gingival Index
5.-Bleeding Index
6.-Probing depths
7.-Attachment levels
8.-Plaque accumulation
9.-Photographs of selected subjects
10.-Dispensing of new tablets and diary
b.-Thirty Day Examinations:
1.-Medical and health update
2.-Oral soft tissue examination
3.-Gingival Index
4.-Bleeding Index
5.-Probing depths
6.-Attachment levels
7.-Plaque accumulation
8.-Photographs of selected subjects
9.-Collection of diary and unused tablets
c-Sixty Day Examinations (Final):
1.-Medical and health update
2.-Oral Soft Tissue examination
3.-Gingival Index
4.-Bleeding Index
5.-Probing depths
6.-Attachment levels
7.-Plaque Accumulation
8.-Photographs of selected subjects

### 2.-Clinical Examinations:

The investigators according to the following evaluation criteria completed, the subject record forms:
a.-Soft Tissue examination and Survey:
   A complete soft and hard tissue examination was performed. The examination included observation of the face, lymph nodes, lips, buccal mucosa, floor of the mouth, tongue, hard palate, soft palate, gingiva and teeth. All findings were recorded as normal or abnormal.
   At each visit, signs of sloughing, erythema, ulceration or edema was looked for. Sloughing is defined as necrotic superficial tissue separating from underlying epithelial tissue, but not extending into the lamina propria. Erythema is defined as a loss of surface necrotic tissue extending into the underlying lamina propria in response to inflammation. Edema is defined as abnormal amounts of fluid in the intercellular spaces, resulting in visible swelling. Each parameter was scored as normal, slight, moderate or marked.
b.-Probing Depth Measurements:
   The probing depth measurements were done according to the method developed by Ramfjord using teeth # 3, 9, 12, 19, 25 and 28. A calibrated periodontal probe was inserted into the pocket with the long axis of the probe aligned parallel to the long axis of the tooth. A measurement was made from the tip of the probe to the level of the gingival margin. The distance was recorded in millimeters and rounded to the nearest whole number.
c.-Gingival Index:
   Gingival Index assessments was done using the method developed by Ramfjord using teeth # 3, 9, 12, 19, 25 and 28 according to the following evaluation criteria:
   0=Absence of inflammation
   1=Mild inflammation—slight change in color and little change in texture
   2=Moderate inflammation—redness, edema glazing and bleeding on probing
   3=Severe inflammation—marked redness and hypertrophy.
d.-Bleeding on Probing:
   Bleeding on probing to the base of the pocket was performed at each evaluation period using the Eastman Bleeding Index according to the following criteria:
   0=No bleeding
   1=Presence of bleeding or a single bleeding point
   2=Interdental triangle is filled with blood
   3=Profuse bleeding, is observed immediately after probing
e.-Clinical Attachment Level:
   To measure clinical attachment level, the cemento-enamel junction (CEJ) was used as a landmark. The end of the probe was manually placed against the enamel surface coronally to the margin of the gingiva on a 45-degree angle to the long axis of the tooth. The probe was then moved in an apical direction and the CEJ is then detected by either tactile sense or by a change in direction of the probe.
   If the gingival margin is on enamel, the distance from the gingival margin to the CEJ was recorded first. Then, the probe was inserted further and the distance from the gingival margin to the bottom of the pocket was then recorded. The first measurement is then subtracted from the second measurement to obtain the clinical attachment level.
   If the gingival margin is on cementum, the clinical attachment level was recorded as a direct measurement from the CEJ to be base of the pocket.
f.-Plaque Index:

A modification of the Ramfjord Plaque Index was used. Plaque is assessed on the facial and lingual surfaces of all the teeth after using a disclosing agent. A plaque score per person was obtained by totaling all of the plaque scores and dividing by the number of surfaces examined. The following criteria were used:
0=No plaque
1=Separate flecks of plaque at the cervical margin of the tooth
2=A thin, continuous band of plaque (up to 1 mm) at the cervical margin.
3=A band of plaque wider than 1 mm but covering less than two thirds of the crown
4=Plaque covering at least one third but less than two thirds of the crown
5=Plaque covering two thirds or more of the crown

Adverse Experience Reporting

All clinically significant changes occurring during or after the treatment, whether or not associated with the treatment were recorded on the case report form. At all evaluation visits, subjects were questioned regarding the occurrence of side effects. The subjects were also asked to contact us with any concerns or unexpected sequella caused by the tablet treatment.

Any adverse experiences which are considered serious are those which result in a life threatening event or death; require hospitalization as a result of the adverse experience; result in a congenital anomaly or malignancy; are the result of an overdose; or result in a permanent disability.

No adverse experience was reported for any of the subjects.

Statistical Analysis

The sample size was estimated with the power analysis, which was performed at a a level of 0.05 and a β level of 0.2 using variance estimates from the literature of 0.3. To detect a change of 0.3 GI or PI units, 15 subjects per group would be required. To detect a change of 0.2 GI units, 30 subjects per group would be required. Again, based on the literature, a realistic expectation for a mean difference between groups is 0.2 units.

The primary analysis of the data was a repeated measure of variance using a 99% level of significance. This statistical model allows comparisons to be made between treatment groups, between time points and between treatment groups at each time point Treatment groups were also be analyzed at post-treatment by analysis of covariance and at baseline using ANOVA.

Results and Discussion

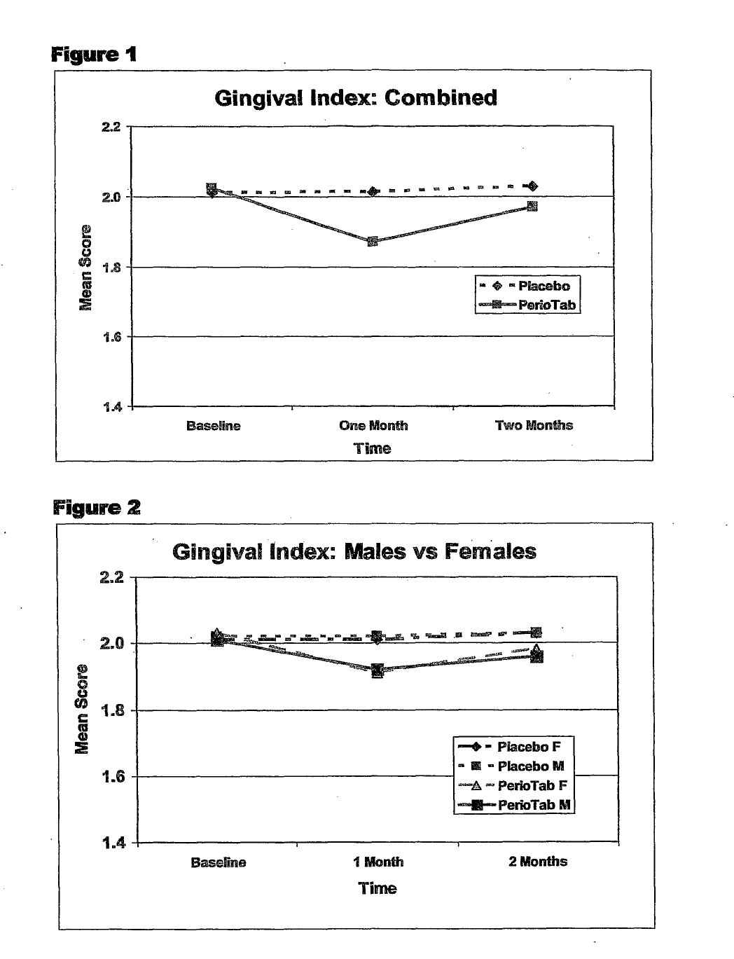

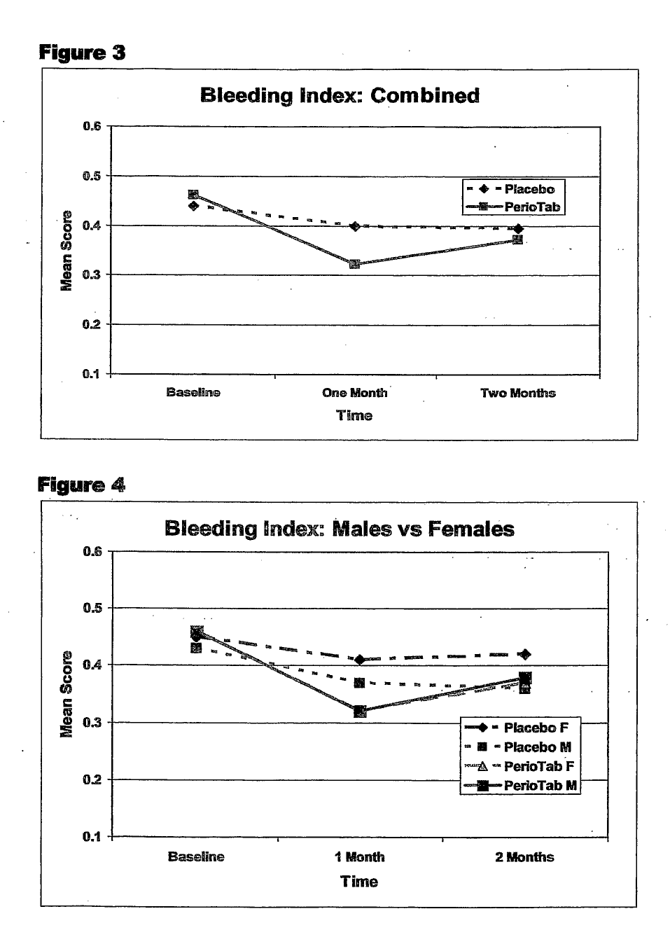

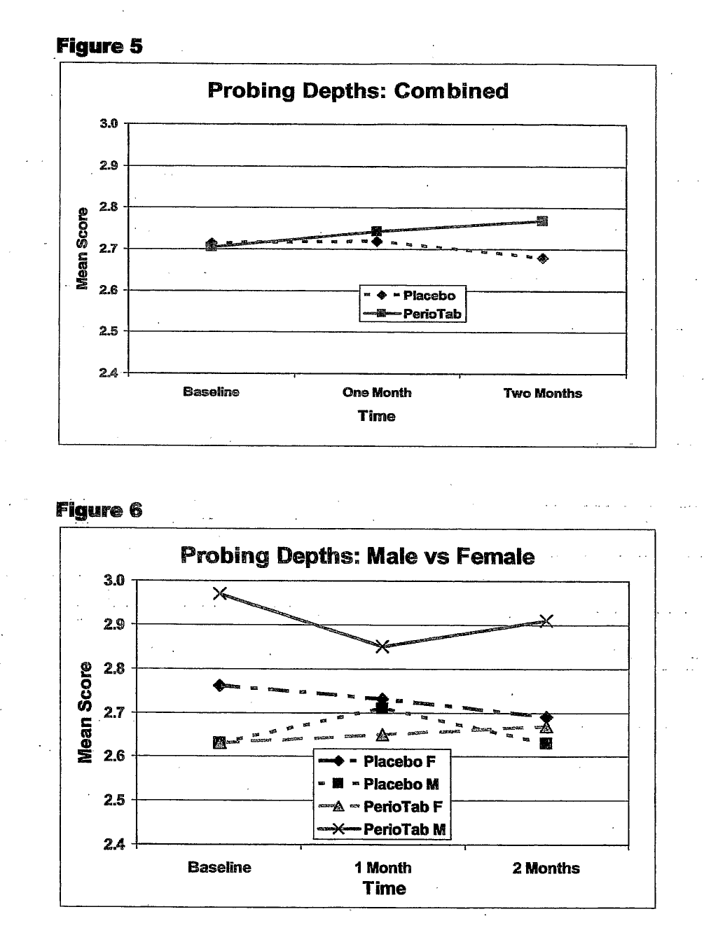

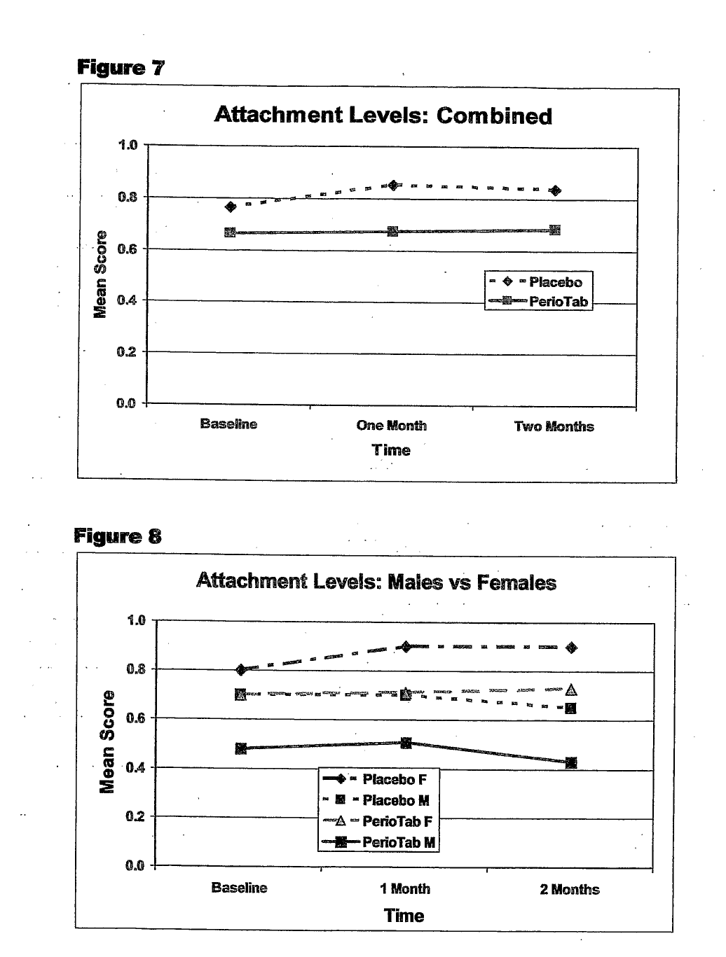

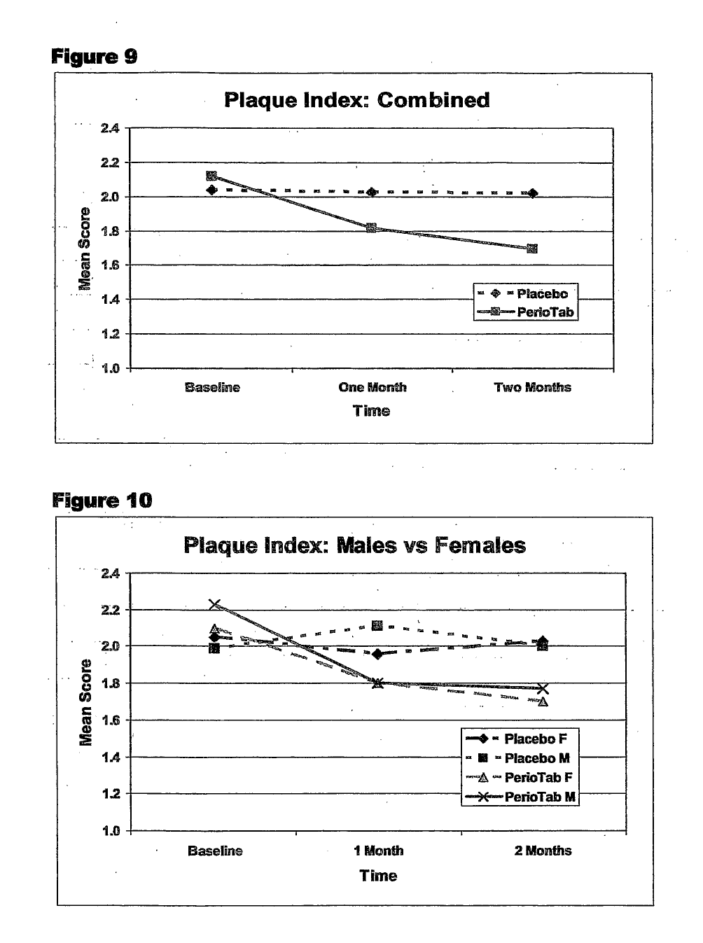

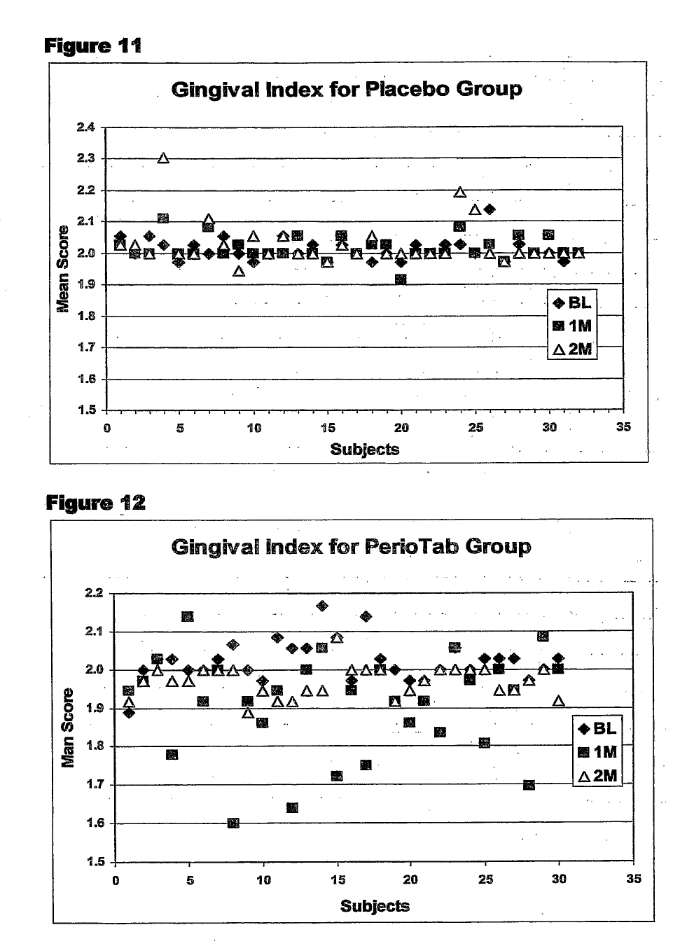

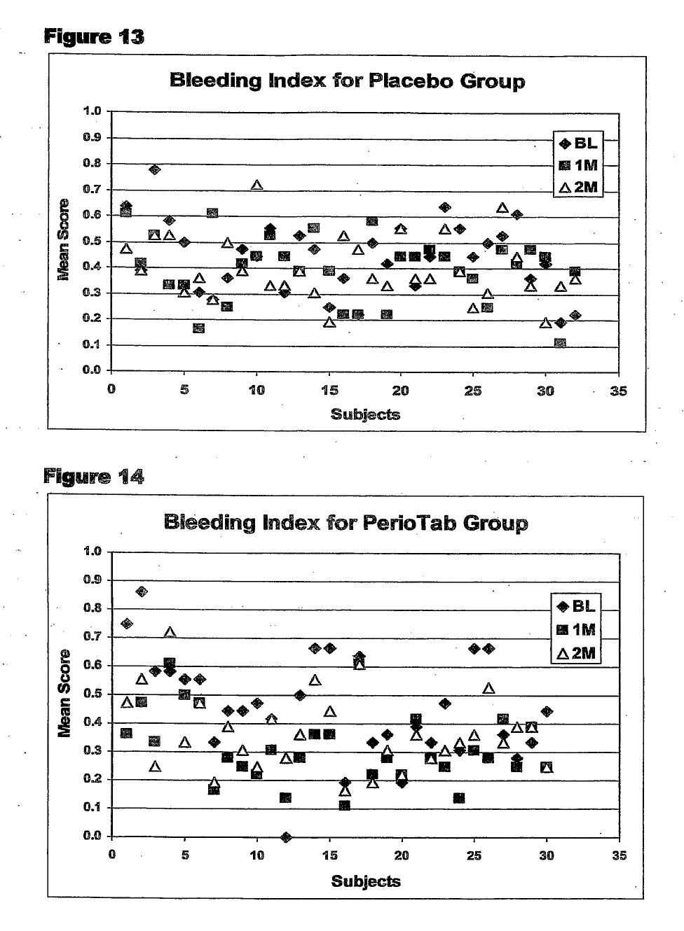

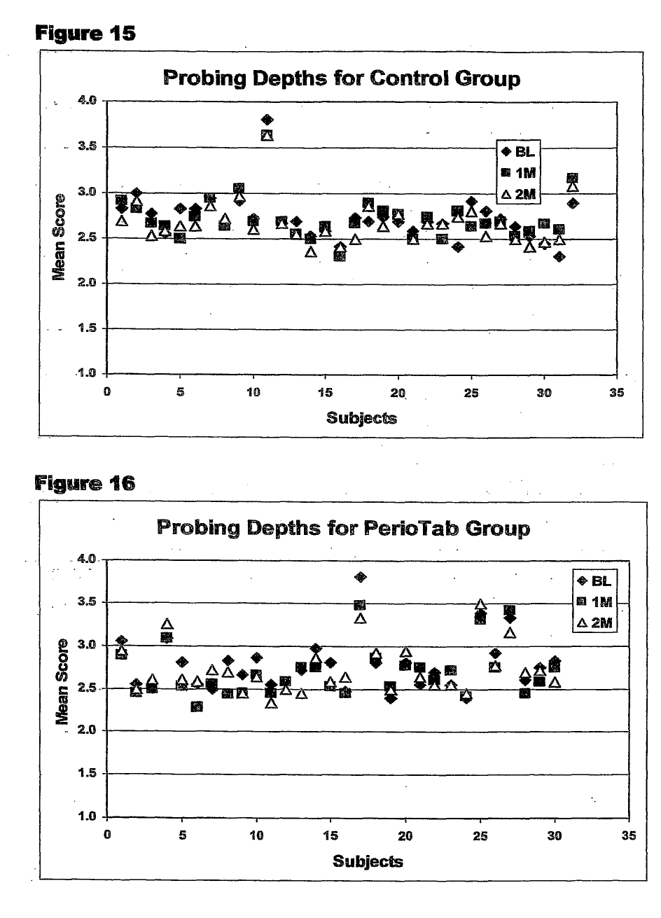

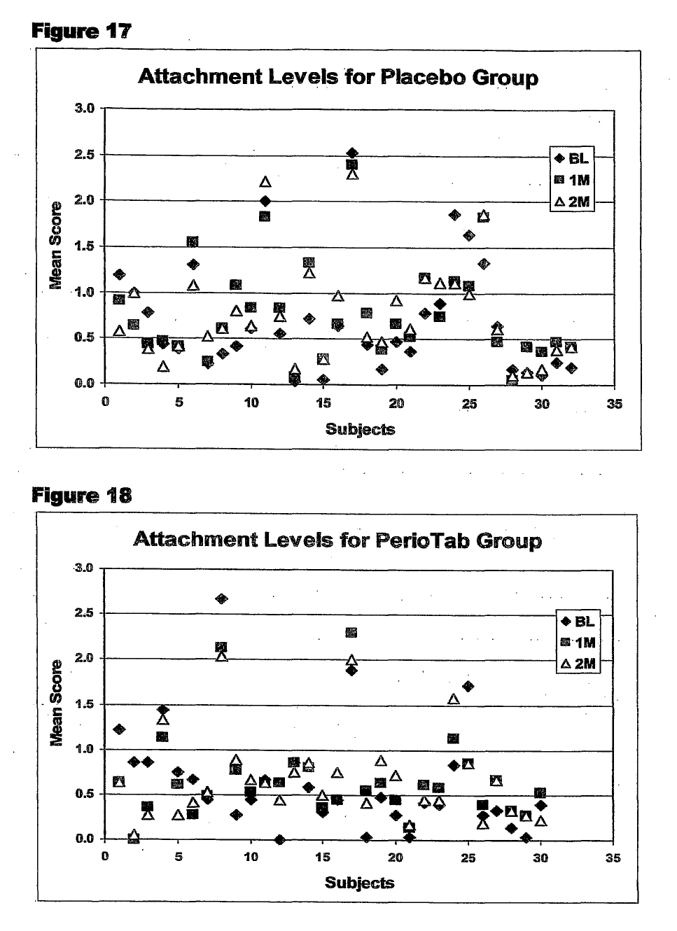

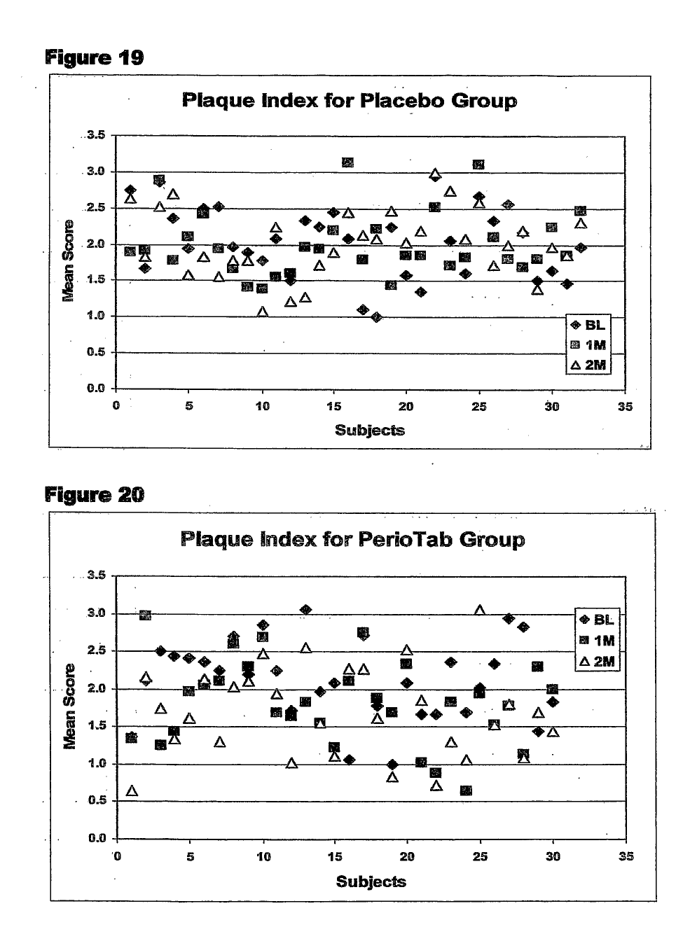

The following clinical parameters were evaluated:
1. Gingival Index (GI)
2. Bleeding Index (BI)
3. Periodontal Probing Depths (PD)
4. Attachment Levels (AL)
5. Plaque Index (PI)
   There were two (2) treatments:
Treatment A=Placebo and
Treatment B=PerioTab (Experimental supplement).
   A total of 33 subjects were randomly assigned to A, and 30 subjects to B.
   The subjects were observed at three (3) time points: Baseline, Seven Days, and 2-months.
   The data is presented in Tables 2-15 and FIGS. 1-20. FIGS. 11-20 shows the mean GI, BI, AL PD and PI for the 62 subjects at each of the three time periods.

All the subjects completed the two-month study except for one subject that withdrew from the study because of family problems.

Every effort was made to ensure the maximum subject compliance. It appeared that the instructions given by the Clinical Coordinator were effective, as subjects usually responded positively at each evaluation time. The greatest complaints were the size and smell/odor of the PerioTab tablet.

Tables 2-4 summarize the results of the GI, BI, PD, PI and AL, at baseline, one month and two months at a significant level of p-value<0.0001. Overall, the data shows a reduction in the Gingival Index, Bleeding Index and Plaque Index, for the PerioTab but no significant changes in the attachment levels and probing depths (Pocket depths). For the placebo group,there, was little reduction in the Gingival Index, Bleeding Index, Probing depths, Attachment level and Plaque Index at any of the time periods. There were no statistically significant changes from baseline to one month to two months for the placebo group.

When the placebo was compared to the PerioTab at each of the three time periods, PerioTab was the most effective at reducing the Gingival Index and Bleeding Index at the one month period and the Gingival Index at the two month evaluation. (p<0.001) (Table 4). There was statistical difference between the two products at the two month-evaluation for the Plaque Index, indicating that PerioTab was more effective at reducing plaque accumulation.

When the data were further separated and analyzed by gender (males vs. females) to determine the effect of the two treatments, PerioTab was more effective than the placebo at reducing the GI, BI, and PI for the females and the Gingival Index for the females. Neither of the two products was effective at reducing the AL and Probing Depths for either males or females (Table 3).

For all the observed treatments, most of the values return to their original score at the two-month evaluation. This seems to indicate that the effect of the PerioTab was diminishing. The subjects took the PerioTab for only seven days as indicated by the sponsor and at the one month-evaluation there was a marked improvement in the Gingival and Bleeding Index. However, at the two-month evaluation the indices had returned to almost the baseline values. This effect shows that the PerioTab needs to be continuously administered to obtain long term results. Perhaps lower doses over a longer period could have shown a more steady improvement.

Due to subjects constraints a non-balanced population was enrolled in the study with fifteen males vs. forty-seven females. Despite the small number of males enrolled in the study, PerioTab showed significant differences for the Gingival and Bleeding indices.

A six to twelve month study needs to be conducted to evaluate the long term effectiveness and safety of PerioTab

### CONCLUSIONS

Under the conditions of the present study, the following conclusions are made.
1.-PerioTab showed significant efficacy in reducing the Gingival, Bleeding and Plaque Indices.
2.-PerioTab is effective at reducing gingivitis and the greatest effect showed at the one month.
3.-Gender has little, impact on the effects of the PerioTab or placebo tablets.
4.-Neither the PerioTab nor the placebo tablet had any effect at increasing the Attachment Levels or decreasing the Pocket Depths in the present study.
5.-There were no additional attachment loss or increase of pocket depths with either the PerioTab or the Placebo during the course of the study.

### REFERENCES

Caton J G, Poison A M (1985). The interdental bleeding index: a simplified procedure for monitoring gingival health. Compend Cont Ed Dent 6: 88, 90-92
Council on Dental Therapeutics (1986). Guidelines for acceptance of chemotherapeutic products for the control of supragingival dental plaque and gingivitis. J Ame Dent Assoc 112: 529-532.
Darby M L, Bowen D M (1983). Research methods for oral health professionals. J.T.K. McCann Company, Pocatello, Id., pp 55-63.
Bensky Dan, Gamble Andrew. Chinese Herbal Medicine Materia Medica; 1993; pgs 64, 142, 227, 329, 334, 340, 342, 345, 347, 349-350, 354, 362, 364, 366, 371, 388, 393-394, 452
Chin Wee Yeow, Keng Hsuan. An Illustrated Dictionary of Chinese Medicinal Herbs. CRCS Publications; 1992
Fawley David O. M. D. Ayurvedic Healing—A Comprehensive Guide. Pgs 106, 193, 337; Morson Publishing; 1992

TABLE 1
Subjects Arranged by Age, race and Gender

| | Male | Female | Totals |
|---|---|---|---|
| Subjects | 16 | 47 | 63 |

Age

| | | |
|---|---|---|
| Oldest | 66 | 63 |
| Youngest | 22 | 25 |

Race

| | | | |
|---|---|---|---|
| Caucasian | 12 | 29 | 40 |
| Hispanic | 3 | 12 | 15 |
| African American | 0 | 3 | 3 |
| Asian | 0 | 3 | 3 |
| Native American | 0 | 0 | 0 |
| Other | 1 | 0 | 1 |
| Totals | 16 | 47 | 63 |

TABLE 2
Comparison for Within Time Periods and Both Genders

| | | | | | | |
|---|---|---|---|---|---|---|
| | BL-1M | | BL-2M | | 1M-2M | |
| Comparisons | P Value | Significance | P Value | Significance | P Value | Significance |

Gingival Index

| | | | | | | |
|---|---|---|---|---|---|---|
| A = Control | 0.596 | No^{c} | 0.240 | No^{c} | 0.989 | No^{c} |
| B = PerioTab | 0.001 | Yes^{a} | 0.001 | Yes^{a} | 0.107 | No^{c} |

Bleeding Index

| | | | | | | |
|---|---|---|---|---|---|---|
| A = Control | 0.196 | No^{c} | 0.198 | No^{c} | 0.978 | No^{c} |
| B = PerioTab | 0.001 | Yes^{a} | 0.030 | Yes^{a} | 0.139 | No^{c} |

Probing Depths

| | | | | | | |
|---|---|---|---|---|---|---|
| A = Control | 0.702 | No^{c} | 0.175 | No^{c} | 0.251 | No^{c} |
| B = PerioTab | 0.188 | No^{c} | 0.544 | No^{c} | 0.501 | No^{c} |

Attachment Levels

| | | | | | | |
|---|---|---|---|---|---|---|
| A = Control | 0.292 | No^{c} | 0.409 | No^{c} | 0.898 | No^{c} |
| B = PerioTab | 0.492 | No^{c} | 0.525 | No^{c} | 0.976 | No^{c} |

Plaque Index

| | | | | | | |
|---|---|---|---|---|---|---|
| A = Control | 0.805 | No^{c} | 0.944 | No^{c} | 0.856 | No^{c} |
| B = PerioTab | 0.035 | Yes^{a} | 0.005 | Yes^{a} | 0.413 | No^{c} |

^{a}= Decrease over time period
b = Increase over time period
^{c}= No change over time period

TABLE 3
Comparison Within Time Periods and Genders

| | | | | | | |
|---|---|---|---|---|---|---|
| Compari- | BL-1M | | BL-2M | Signifi- | 1M-2M | Signifi- |
| sons | P Value | Significance | P Value | cance | P Value | cance |

Gingival Index

| | | | | | | |
|---|---|---|---|---|---|---|
| Control | | | | | | |
| Females | 0.886 | No^{c} | 0.684 | No^{c} | 0.775 | No^{c} |
| Males | 0.535 | No^{c} | 0.894 | No^{c} | 0.756 | No^{c} |
| PerioTab | | | | | | |
| Females | 0.001 | Yes^{a} | 0.001 | Yes^{a} | 0.078 | No^{c} |
| Males | 0.152 | No^{c} | 0.012 | Yes^{a} | 0.506 | No^{c} |

Bleeding Index

| | | | | | | |
|---|---|---|---|---|---|---|
| Control | | | | | | |
| Females | 0.352 | No^{c} | 0.381 | No^{c} | 0.922 | No^{c} |
| Males | 0.359 | No^{c} | 0.327 | No^{c} | 0.916 | No^{c} |
| PerioTab | | | | | | |
| Females | 0.005 | Yes^{a} | 0.922 | No^{c} | 0.252 | No^{c} |
| Males | 0.086 | No^{c} | 0.916 | No^{c} | 0.232 | No^{c} |

Probing Depths

| | | | | | | |
|---|---|---|---|---|---|---|
| Control | | | | | | |
| Females | 0.531 | No^{c} | 0.116 | No^{c} | 0.435 | No^{c} |
| Males | 0.435 | No^{c} | 0.953 | No^{c} | 0.467 | No^{c} |
| PerioTab | | | | | | |
| Females | 0.274 | No^{c} | 0.695 | No^{c} | 0.536 | No^{c} |
| Males | 0.649 | No^{c} | 0.745 | No^{c} | 0.818 | No^{c} |

Attachment Levels

| | | | | | | |
|---|---|---|---|---|---|---|
| Control | | | | | | |
| Females | 0.277 | No^{c} | 0.282 | No^{c} | 0.982 | No^{c} |
| Males | 0.724 | No^{c} | 0.851 | No^{c} | 0.724 | No^{c} |
| PerioTab | | | | | | |
| Females | 0.789 | No^{c} | 0.585 | No^{c} | 0.845 | No^{c} |
| Males | 0.240 | No^{c} | 0.818 | No^{c} | 0.575 | No^{c} |

Plaque Index

| | | | | | | |
|---|---|---|---|---|---|---|
| Control | | | | | | |
| Females | 0.554 | No^{c} | 0.544 | No^{c} | 0.648 | No^{c} |
| Males | 0.596 | No^{c} | 0.912 | No^{c} | 0.632 | No^{c} |
| PerioTab | | | | | | |
| Females | 0.108 | No^{c} | 0.013 | Yes^{a} | 0.385 | No^{c} |
| Males | 0.149 | No^{c} | 0.149 | No^{c} | 0.967 | No^{c} |

^{a}= Decrease over time period
b = Increase over time period
^{c}= No change over time period

TABLE 4
Comparisons Between Treatments and Time

| | | | | | | |
|---|---|---|---|---|---|---|
| | | | One | | Two | |
| | Baseline | | Month | | Months | |
| Comparisons | P Value | Significance | P Value | Significance | P Value | Significance |

Gingival Index

| | | | | | | |
|---|---|---|---|---|---|---|
| Control vs | 0.334 | No^{d} | 0.001 | Yes^{e} | 0.001 | Yes^{e} |
| PerioTab | | | | | | |

Bleeding Index

| | | | | | | |
|---|---|---|---|---|---|---|
| Control vs | 0.675 | No^{d} | 0.014 | Yes^{e} | 0.254 | No^{d} |
| PerioTab | | | | | | |

Probing Depths

| | | | | | | |
|---|---|---|---|---|---|---|
| Control vs | 0.767 | No^{d} | 0.231 | No^{d} | 0.698 | No^{d} |
| PerioTab | | | | | | |

Attachment Levels

| | | | | | | |
|---|---|---|---|---|---|---|
| Control vs | 0.757 | No^{d} | 0.394 | No^{d} | 0.486 | No^{d} |
| PerioTab | | | | | | |

Plaque Index

| | | | | | | |
|---|---|---|---|---|---|---|
| Control vs | 0.505 | No^{d} | 0.147 | No^{d} | 0.017 | Yes^{e} |
| PerioTab | | | | | | |

^{d}= No difference between Control and PerioTab
^{e}= Control Higher than PerioTab
f = PerioTab higher than Control

TABLE 5
Descriptive Statistics of the Difference
in Means for the GI, BI, PD, AL and PI

| | | |
|---|---|---|
| BL-1M | BL-2M | 1M-2M |

Gingival Index

| | | | |
|---|---|---|---|
| Control | −0.004 | −0.017 | −0.013 |
| PerioTab | 0.113 | 0.054 | −0.059 |

Bleeding Index

| | | | |
|---|---|---|---|
| Control | 0.044 | 0.043 | −0.001 |
| PerioTab | 0.142 | 0.092 | −0.005 |

Probing Depths

| | | | |
|---|---|---|---|
| Control | 0.001 | 0.048 | 0.047 |
| PerioTab | 0.004 | −0.029 | −0.033 |

Attachment Levels

| | | | |
|---|---|---|---|
| Control | −0.078 | −0.666 | 0.012 |
| PerioTab | −0.015 | −0.019 | −0.004 |

Plaque Index

| | | | |
|---|---|---|---|
| Control | 0.029 | 0.008 | −0.021 |
| PerioTab | 0.036 | 0.429 | 0.123 |

Negative numbers represent an increase in the values

TABLE 6
Gingival Index: Descriptive Statistics
for Males and Females Combined

| | | n | Mean | SD |
|---|---|---|---|---|
| Tx | Control | 3456 | 2.02 | 0.02 |
| | PerioTab | 3240 | 1.97 | 0.05 |
| Time | Baseline | 2220 | 2.02 | 0.04 |
| | 1-month | 2220 | 1.96 | 0.08 |
| | 2-month | 2220 | 2.00 | 0.06 |
| Tx(Time) | Control (BL) | 1146 | 2.01 | 0.04 |
| | PerioTab(BL) | 1074 | 2.02 | 0.05 |
| | Control (1-mon) | 1146 | 2.02 | 0.04 |
| | PerioTab (1-mon) | 1074 | 1.91 | 0.13 |
| | Control (2-mon) | 1146 | 2.03 | 0.07 |
| | PerioTab (2-mon) | 1074 | 1.97 | 0.04 |

TABLE 7
Gingival Index: Descriptive Statistics for Males vs females

| Tx(Time) | | n | Mean | SD |
|---|---|---|---|---|
| Females: | Control (BL) | 822 | 2.01 | 0.03 |
| | PerioTab(BL) | 858 | 2.03 | 0.06 |
| | Control (1-mon) | 822 | 2.01 | 0.04 |
| | PerioTab (1-mon) | 858 | 1.91 | 0.13 |
| | Control (2-mon) | 822 | 2.03 | 0.07 |
| | PerioTab (2-mon) | 858 | 1.98 | 0.04 |
| Males: | Control (BL) | 324 | 2.02 | 0.05 |
| | PerioTab(BL) | 216 | 2.01 | 0.02 |
| | Control (1-mon) | 324 | 2.02 | 0.04 |
| | PerioTab (1-mon) | 216 | 1.92 | 0.14 |
| | Control (2-mon) | 324 | 2.03 | 0.08 |
| | PerioTab (2-mon) | 216 | 1.96 | 0.03 |

TABLE 8
Bleeding Index: Descriptive Statistics
for Males and Females Combined

| | | n | Mean | SD |
|---|---|---|---|---|
| Tx | Control | 1431 | 0.41 | 0.03 |
| | PerioTab | 1273 | 0.38 | 0.03 |
| Time | Baseline | 1043 | 0.45 | 0.16 |
| | 1-month | 803 | 0.36 | 0.13 |
| | 2-month | 858 | 0.38 | 0.13 |
| Tx(Time) | Control (BL) | 510 | 0.44 | 0.14 |
| | PerioTab(BL) | 533 | 0.46 | 0.19 |
| | Control (1-mon) | 460 | 0.40 | 0.13 |
| | PerioTab (1-mon) | 343 | 0.32 | 0.13 |
| | Control (2-mon) | 461 | 0.40 | 0.12 |
| | PerioTab (2-mon) | 397 | 0.37 | 0.13 |

TABLE 9
Bleeding Index: Descriptive Statistics for Males vs females

| Tx(Time) | | n | Mean | SD |
|---|---|---|---|---|
| Females: | Control (BL) | 372 | 0.45 | 0.14 |
| | PerioTab(BL) | 434 | 0.46 | 0.19 |
| | Control (1-mon) | 341 | 0.41 | 0.19 |
| | PerioTab (1-mon) | 275 | 0.32 | 0.14 |
| | Control (2-mon) | 344 | 0.42 | 0.12 |
| | PerioTab (2-mon) | 316 | 0.37 | 0.14 |
| Males: | Control (BL) | 138 | 0.43 | 0.14 |
| | PerioTab(BL) | 99 | 0.46 | 0.17 |
| | Control (1-mon) | 119 | 0.37 | 0.12 |
| | PerioTab (1-mon) | 68 | 0.32 | 0.07 |
| | Control (2-mon) | 117 | 0.36 | 0.13 |
| | PerioTab (2-mon) | 81 | 0.37 | 0.09 |

TABLE 10
Pocket Depths: Descriptive Statistics
for Males and females combined

| | | n | Mean | SD |
|---|---|---|---|---|
| Tx | Control | 3438 | 2.37 | 0.01 |
| | PerioTab | 3220 | 2.70 | 0.18 |
| Time | Baseline | 2220 | 2.71 | 0.43 |
| | 1-month | 2220 | 2.71 | 0.27 |
| | 2-month | 2220 | 2.70 | 0.26 |
| Tx(Time) | Control (BL) | 1146 | 2.73 | 0.26 |
| | PerioTab(BL) | 1074 | 2.69 | 0.60 |
| | Control (1-mon) | 1146 | 2.73 | 0.24 |
| | PerioTab (1-mon) | 1074 | 2.69 | 0.30 |
| | Control (2-mon) | 1146 | 2.68 | 0.24 |
| | PerioTab (2-mon) | 1074 | 2.68 | 0.49 |

TABLE 11
Pocket Depths: Descriptive Statistics for Males vs females

| Tx(Time) | | n | Mean | SD |
|---|---|---|---|---|
| Females | Control (BL) | 822 | 2.77 | 0.27 |
| | PerioTab(BL) | 858 | 2.63 | 0.63 |
| | Control (1-mon) | 822 | 2.73 | 0.26 |
| | PerioTab (1-mon) | 858 | 2.64 | 0.26 |
| | Control (2-mon) | 822 | 2.70 | 0.26 |
| | PerioTab (2-mon) | 858 | 2.67 | 0.25 |
| Males | Control (BL) | 324 | 2.63 | 0.22 |
| | PerioTab(BL) | 216 | 2.97 | 0.32 |
| | Control (1-mon) | 324 | 2.71 | 0.19 |
| | PerioTab (1-mon) | 216 | 2.87 | 0.41 |
| | Control (2-mon) | 324 | 2.64 | 0.21 |
| | PerioTab (2-mon) | 216 | 2.91 | 0.36 |

TABLE 12
Attachment Levels: Descriptive Statistics
for Males and Females Combined

| | | n | Mean | SD |
|---|---|---|---|---|
| Tx | Control | 3438 | 0.82 | 0.04 |
| | PerioTab | 3220 | 0.67 | 0.07 |
| Time | Baseline | 2220 | 0.71 | 0.74 |
| | 1-month | 2220 | 0.76 | 0.64 |
| | 2-month | 2220 | 0.76 | 0.64 |
| Tx(Time) | Control (BL) | 1146 | 0.77 | 0.05 |
| | PerioTab(BL) | 1074 | 0.66 | 0.61 |
| | Control (1-mon) | 1146 | 0.85 | 0.79 |
| | PerioTab (1-mon) | 1074 | 0.67 | 0.49 |
| | Control (2-mon) | 1146 | 0.84 | 0.79 |
| | PerioTab (2-mon) | 1074 | 0.68 | 0.49 |

TABLE 13
Attachment Levels: Descriptive Statistics for Males vs Females

| Tx(Time) | | n | Mean | SD |
|---|---|---|---|---|
| Females: | Control (BL) | 822 | 0.81 | 0.93 |
| | PerioTab(BL) | 858 | 0.70 | 0.61 |
| | Control (1-mon) | 822 | 0.91 | 0.90 |
| | PerioTab (1-mon) | 858 | 0.71 | 0.53 |
| | Control (2-mon) | 822 | 0.91 | 0.86 |
| | PerioTab (2-mon) | 858 | 0.74 | 0.52 |
| Males: | Control (BL) | 324 | 0.70 | 0.71 |
| | PerioTab(BL) | 216 | 0.49 | 0.62 |
| | Control (1-mon) | 324 | 0.69 | 0.55 |
| | PerioTab (1-mon) | 216 | 0.51 | 0.22 |
| | Control (2-mon) | 324 | 0.65 | 0.58 |
| | PerioTab (2-mon) | 216 | 0.43 | 0.27 |

TABLE 14
Plaque Accumulation: Descriptive Statistics
for Males and Females Combined

| | | n | Mean | SD |
|---|---|---|---|---|
| Tx | Control | 3432 | 2.02 | 0.03 |
| | PerioTab | 3222 | 1.88 | 0.03 |
| Time | Baseline | 2214 | 2.08 | 0.52 |
| | 1-month | 2220 | 1.91 | 0.50 |
| | 2-month | 2220 | 1.86 | 0.53 |
| Tx(Time) | Control (BL) | 1140 | 2.04 | 0.50 |
| | PerioTab(BL) | 1074 | 2.12 | 0.53 |
| | Control (1-mon) | 1146 | 2.01 | 0.44 |
| | PerioTab (1-mon) | 1074 | 1.82 | 0.56 |
| | Control (2-mon) | 1146 | 2.03 | 0.47 |
| | PerioTab (2-mon) | 1074 | 1.70 | 0.60 |

TABLE 15
Plaque Accumulation: Descriptive Statistics for Males vs Females

| Tx(Time) | | n | Mean | SD |
|---|---|---|---|---|
| Females: | Control (BL) | 816 | 2.14 | 0.52 |
| | PerioTab(BL) | 858 | 2.16 | 0.53 |
| | Control (1-mon) | 822 | 1.97 | 0.44 |
| | PerioTab (1-mon) | 858 | 1.83 | 0.60 |
| | Control (2-mon) | 822 | 2.03 | 0.52 |
| | PerioTab (2-mon) | 858 | 1.68 | 0.58 |
| Males: | Control (BL) | 324 | 1.99 | 0.46 |
| | PerioTab(BL) | 216 | 2.24 | 0.58 |
| | Control (1-mon) | 324 | 2.11 | 0.45 |
| | PerioTab (1-mon) | 216 | 1.78 | 0.41 |
| | Control (2-mon) | 324 | 2.01 | 0.34 |
| | PerioTab (2-mon) | 216 | 1.77 | 0.68 |

1. Composition comprising a mixture of radix polygoni multiflori, Fructus Corni, cuscuta japonica, rehmannia glutinosa, licorice, asparagine, Derla andrographis.

2. Composition comprising a mixture of Salvida persica, achyranthes aspera, spilanthes acmela, clove, picus Bengalensis, acacia nilotioca resen, eucalyptus, mint, green tea, bamboo silica.

3. Composition according to claim 1, further comprising a composition comprising a mixture of Salvida persica, achyranthes aspera, spilanthes acmela, clove, picus Bengalensis, acacia nilotioca resen, eucalyptus, mint, green tea, bamboo silica.

4. Composition according to claim 1, further comprising additional components selected from the group consisting of carriers, thickening materials, humectants, water, buffering agents, abrasive polishing materials, surfactants, titanium dioxide, flavor system, sweetening agents, coloring agents, antioxidants, adjuvants, preservatives, stabilisers, homogenising agents, texturising agents, soothing agents and mixtures thereof.

5. Oral hygiene product, comprising the composition of claim 1.

6. The composition of claim 1, wherein the composition has an anti-microbial effect on a cariogenic bacterium and/or a micro-organism selected from the group consisting of Gram positive bacteria, Gram negative bacteria, S. mutans, S. sobrinus, L. acidophilus, L, casei, L. plantarum, A. naeslundii, A. viscosus, Actinobacillus actinomycetemcomitants, Porphyromonas gingivalis, Fusobacteriam nucleaium, Treponema denticola, Bacteroides forsythus, Candidas albicans, C. glabrata, C, guilliemondii, C. kefyr, C. krusei, C. stellaioidea, C. tropicalis.

7. Use of a composition according to claim 1, as a medicament for the acute and/or long term treatment of microbial infections, in particular of oral pathogenic micro-organisms, in particular dental caries, periodontosis, gingivitis, gum disease, gum bleeding and/or plaque reduction.

8. Use of a composition according to claim 1, in a method for the preparation of a medicament for the acute and/or long term treatment of microbial infections, in particular of oral pathogenic micro-organisms, in particular dental caries, periodontosis, gingivitis, gum disease, gum bleeding and/or plaque reduction.

9. Method for the preparation of an extract from the components as defined in claim 1, comprising supercritical carbon dioxide liquid extraction.

10. Dried extraction elute of the composition of claim 1.

11. Composition according to claim 2, further comprising additional components selected from the group consisting of earners, thickening materials, humectants, water, buffering agents, abrasive polishing materials, surfactants, titanium dioxide, flavor system, sweetening agents, coloring agents, antioxidants, adjuvants, preservatives, stabilisers, homogenising agents, texturising agents, soothing agents and mixtures thereof.

12. Oral hygiene product, comprising the composition of claim 2.

13. The composition of claim 2, wherein the composition has an anti-microbial effect on a cariogenic bacterium and/or a micro-organism selected from the group consisting of Gram positive bacteria, Gram negative bacteria, S. mutans, S. sobrinus, L. acidophilus, L. casei, L. plantarum, A. naeslundii, A. viscosus, Actinobacillus actinomycetemcomitants, Porphyromonas gingivalis, Fusobacterium nucleatum, Treponema denticola, Bacteroides forsythus, Candidas albicans, C. glabrata, C. guilliemondii, C. kejyr, C. krusei, C. stellatoidea, C. tropicalis.

14. Use of a composition according to claim 2, as a medicament for the acute and/or long term treatment of microbial infections, in particular of oral pathogenic micro-organisms, in particular dental caries, periodontosis, gingivitis, gum disease, gum bleeding and/or plaque reduction.

15. Use of a composition according to claim 2, in a method for the preparation of a medicament for the acute and/or long term treatment of microbial infections, in particular of oral pathogenic micro-organisms, in particular dental caries, periodontosis, gingivitis, gum disease, gum bleeding and/or plaque reduction.

16. Method for the preparation of an extract from the components as defined in claim 2, comprising supercritical carbon dioxide liquid extraction.

17. Dried extraction elute of the composition of claim 2.
